(19) United States
(12) Patent Application Publication
(10) Pub. No.: US 2014/0357910 A1
(43) Pub. Date: Dec. 4, 2014

(54) SURFACE MODIFICATION OF CATALYSTIC SURFACE BY ORGANIC MOLECULES AND METAL CATIONS FOR SELECTIVE CATALYSIS

(71) Applicants: Elena Shevchenko, Riverside, IL (US); Soongu Kwon, Woodridge, IL (US); Emilio Bunel, Naperville, IL (US); Galyna Krylova, South Bend, IL (US); Julius Jellinek, Chicago, IL (US); Aslihan Sumer, Woodridge, IL (US); Christopher L. Marshall, Naperville, IL (US)

(72) Inventors: Elena Shevchenko, Riverside, IL (US); Soongu Kwon, Woodridge, IL (US); Emilio Bunel, Naperville, IL (US); Galyna Krylova, South Bend, IL (US); Julius Jellinek, Chicago, IL (US); Aslihan Sumer, Woodridge, IL (US); Christopher L. Marshall, Naperville, IL (US)

(21) Appl. No.: 13/905,452

(22) Filed: May 30, 2013

(51) Int. Cl. B01J 31/02; C07C 5/09

(52) U.S. Cl. CPC B01J 31/0238; C07C 5/09
USPC 585/273; 502/167

(57) ABSTRACT

An article and method of manufacture of a catalyst. The article includes a nanoparticle of a noble metal based on material with a primary alkylamine layer disposed on the surface of the nanoparticle catalyst. The alkylamine layer of at least about one monolayer establishes a minimum level of selectivity for hydrogenation reactions.

### STATEMENT OF GOVERNMENT INTEREST

The United States Government has certain rights in this invention pursuant to Contract No. W-31-109-ENG-38 between the United States Government and The University of Chicago and/or pursuant to Contract No. DE-AC02-06CH11357 between the United States Government and UChicago Argonne, LLC representing Argonne National Laboratory.

### FIELD OF THE INVENTION

This invention relates generally to a method and an article of manufacture for selective surface modification of nanoparticle catalysts for use in alkyne hydrogenation reactions. More particularly the invention relates to a method and article of manufacture for producing catalysts which provide a balance between absorption energetics of alkenes at the surface of nanoparticles as compared to nonselective capping ligands to thereby provide highly selective nanocatalysts for alkene in alkene hydrogenation reactions.

### BACKGROUND OF THE INVENTION

The field of catalysis has substantial commercial importance in production of various chemicals. In addition, the need to perform chemical processes with reduced pollution for “green chemistries” emphasizes the importance of catalysts with improved selectivity since they can reduce significantly the amount of generated chemical waste and pollution. High-performance catalysts also play an important role in energy conversion and storage technologies. Moreover, nanocatalysts can provide an improved group of catalytic materials. Significant progress has been made in the synthesis of high performance nanocatalysts using solution-based approaches. Thus the specific activity of nanocatalysts (hereinafter “NPs”) can be greatly improved by the decrease in their particle size and by tuning their composition and selective surface treatment. Catalytically active NPs with certain shapes can also dramatically affect the reaction pathways and change the selectivity of reactions. For example, Pt based NPs with high-index facets prepared electrochemically have very high catalytic activity in the electro-oxidation of formic acid and ethanol. Also high surface-to-volume ratio of Pd—Pt nanodendrites can lead to enhanced electrocatalytic activity. Control of the size and shape of NPs is often achieved by the introduction of certain surfactant molecules that play a key role in synthesis of NPs. These molecules form complexes with the precursors and bind onto the surface of NPs affecting their nucleation and growth processes. As a result, the surface of the NPs synthesized in solution is covered by a layer (fill or partial) of capping ligand molecules. These adsorbed molecules can significantly impact the performance of nanocatalysts since catalytic reactions take place at the surface, and ligands can affect the electronic characteristics of surface sites as well as hinder the access of the substrate molecules to the surface of the NPs.

The role of capping ligands in catalytic reactions, wherein chemically synthesized nanocatalysts are used, is still not at all well understood. Both moderate and reduced catalytic activities have been reported for surfactant-stabilized NPs. Surface molecules on colloidal NPs are usually remnants of chemical compounds introduced into reaction mixtures. Modification of metal surfaces (e.g., Pt, Pd, and others) by the simple addition of strongly adsorbing chiral molecules has allowed for efficient stereoselective control over reactions at the metal surface. In spite of the extensive research there has not been developed an adequate understanding to enable preparation of any reliably selective catalysts.

### SUMMARY OF THE INVENTION

Nanoparticles catalysts with modified surface structures have been prepared and preferably for use in alkyne hydrogenation reactions. The effect of surtface ligands on the selectivity and activity of Pt and Co/Pt based nanoparticles (NPs) have been determined and evaluated using experimental and computational approaches. A proper balance between adsorption energetics of alkenes at the surface of NPs as compared to that of capping ligands can define the selectivity of the nanocatalyst for alkene in alkyne hydrogenation reaction. Addition of primary alkylamines to Pt and CoPt₃ NPs can substantially increase selectivity for alkene from virtually 0 to more than 90% with about 99.9% conversion. Increasing the primary alkylamine coverage on the NP surface can lead to the decrease in the binding energy of octenes and eventual competition between octene and primary alkylamines for adsorption sites. At sufficiently high coverage of catalysts with primary alkylamine, the alkylamines prevail, which prevents further hydrogenation of alkenes into alkanes. Primary amines with different lengths of carbon chains have similar adsorption energies at the surface of catalysts and consequently have the same effect on selectivity. When the adsorption energy of capping ligands at the catalytic surface is lower than adsorption energy of alkenes, the ligands do not affect the selectivity of hydrogenation of alkyne to alkene. On the other hand, capping ligands with adsorption energies at the catalytic surface higher than that of alkyne reduce its activity, thereby resulting in low conversion of alkynes.

Various aspects of the invention are described hereinafter; and these and other objects of improvements are described in detail hereinafter, including the drawings described in the following section.

### BRIEF DESCRIPTION OF THE DRAWINGS

FIG. 1a illustrates a TEM image of 3.5 mm Pt nanoparticles with a concentration of 2.6±0.5 mM of Pt; FIG. 1b illustrates a TEM image of 4.5 mm Co Pt₃ nanoparticles with a concentration of 2.6±0.5 mM of Pt; and FIG. 1c illustrates the composition of reaction products formed as a result of 4-octane hydrogenation in solution containing only clean NPs with 0.13 M of 1-octylamine;

FIG. 2 illustrates a plot of percent composition for a series of different capping ligands, showing the effect of different capping ligands on activity and selectivity of 3.5 nm Pt NPs in hydrogenation reactions of 4-octyne and the concentration of all ligands was about 23 mM;

FIG. 3a shows percent selectivity and conversion of 4-octyne hydrogenation at different concentrations of octylamine wherein the concentration of Pt was about 3 mM; FIG. 3b shows amine concentration-dependent percent selectivity for different [Pt]_{surf} as shown in the plot; FIG. 3c shows composition of 4-octene in product from trans-/cis-4-octene with relative amounts of alkene defined as [alkene]normalized to an initial concentration of alkene or alkync, and the inset plot shows conversion percent for 4-octene hydrogenation defined as 1-[4-octene]_{final}/[4-octene]ᵢₙᵢₜᵢₐₗ;

FIG. 4a shows plots of adsorption energies (E_{ads}) of 4-octyne, cis-4-octene, trans-4-octene and 1-octylamine on clean and 1-octylamine decorated (111) Pt surface (as represented by a 35 atom Pt cluster) as a function of octylamine coverage with adsorption energies obtained from state-of-the-art density functional theory (DFT) computations (the dashed circle highlights diftirence in E, of 4-octene and octylamine), and FIG. 4b shows depictions of optimized structures obtained in DFT calculations;

FIG. 5 shows kinetic data which demonstrate conversion (see upper graph section) and selectivity (lower section) of 4-octyne hydrogenation using 3.5 nm Pt NPs as a function of octylamine concentration percentage (concentration of Pt was 2.6±0.5 mM and data points denotes “r” and “b” represent low and high selectivity regimes, respectively)

FIG. 6 illustrates a summary of DFT resultsipredictions on effects of position of the C═C bond and/or length of carbon chain on adsorption energies of alkenes;

FIG. 7 shows selectivity in hydrogenation reactions of different alkyne molecules with reactions performed with a fixed concentration for I-octylamine at 39 nM using 3.5 nm Pt NPs and Pt concentrations of 2.6±0.5 mM;

FIG. 8a shows a summary of DFT results on adsorption energies of primary amines with different carbon chain lengths; FIG. 8b shows effect of carbon chain length in primary amines on selectivity in 4-octyne hydrogenation reactions using 3.5 nm Pt NPs and the terms C₆, C₈, C₁₂ and C₁₈ are abbreviations for 1-hexyl, 1-octyl; 1-dodecyl-, and 1-octadecylamine, respectively, with amine concentrations of 39 mM and Pt of 2.6±0.5 mM; and

FIG. 9 shows selectivity as a function of free amine concentration in the solution (curve “v”) and the fitting curve calculated using Eqn. 2 (curve “r”) with the data from FIG. 3a.

### DETAILED DESCRIPTION OF PREFERRED EMBODIMENTS

In a preferred form of the invention, selected capping ligands, such as primary amines were determined to be highly selective versus other well-known capping ligands, such as trioctylphosphine (TOP), trioctylphosphine oxide (TOPO), and oleic acid. The catalytic functionality was shown by examples using treated Pt and Co/Pt NPs in hydrogenation of alkynes. Selective hydrogenation of alkynes into alkenes has been an important commercial area in catalytic hydrogenation reactions. The addition of primary alkylamines to Pt and CoPt3 NPs can drastically increase the selectivity for alkene from about 0 to >90% without affecting the catalytic activity. A computational analysis is provided for the observed experimental effects of the primary amine on the selectivity and activity of the NPs. Also provided is a general understanding of the effect of surface modification of NPs on selectivity and conversion in hydrogenation of alkynes. In particular, the source of these effects was determined to be the ligand coverage dependent difference in the adsorption energetics of the substrate molecules and certain very particular modifier ligands.

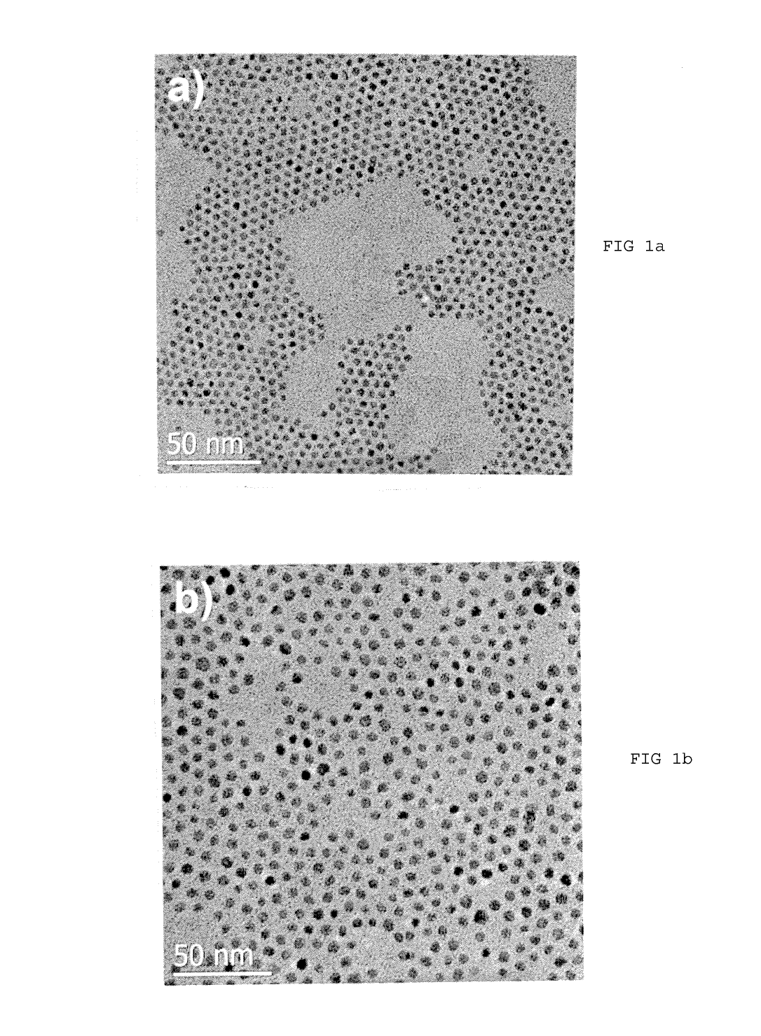

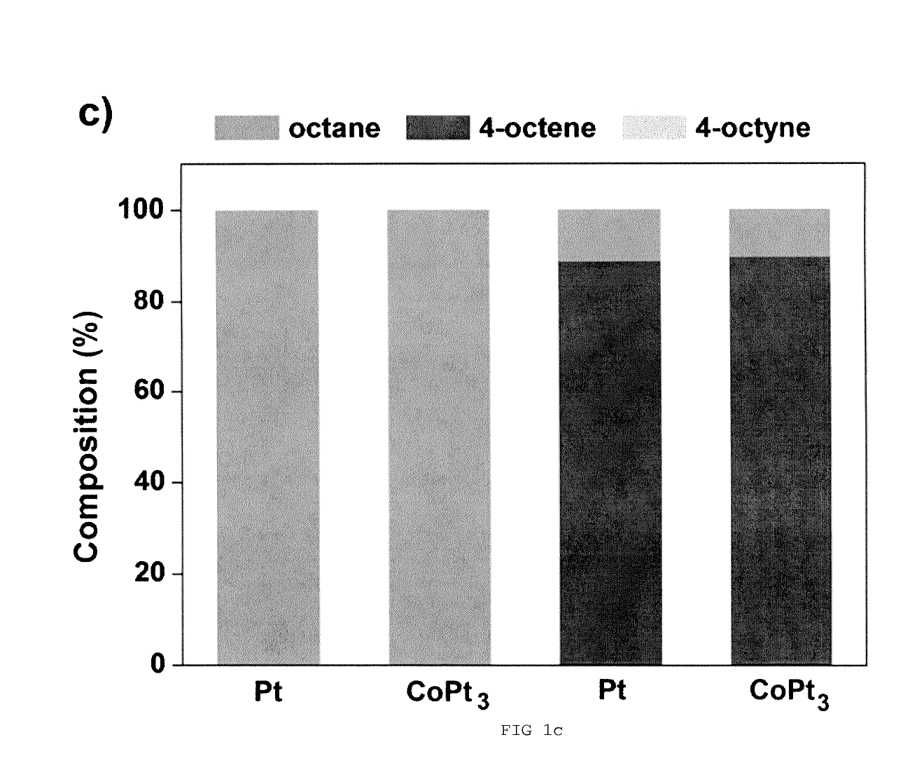

In the evaluation 3.5 nm Pt and 4.5 nm alloyed CoPt₃ NPs (FIGS. 1a and 1b) were chosen as model catalysts based on good catalytic activity of Pt in hydrogenation reactions. While these illustrate the concept of the invention, it is understood that other typical catalysts, like Pd based materials, can also benefit from the instant invention. In the preferred embodiments the Pt and CoPt₃ NPs were synthesized according to conventional, well known procedures. After synthesis, NPs were thoroughly purified by repeated washing and precipitation. According to thermogravimetric analysis (TGA), the amount of residual alkylamine ligand on purified Pt NPs was about 4 wt % that corresponds to 0.1 mM in the hydrogenation reaction solution. The hydrogenation reactions were performed using a stainless steel reactor under 200 psig H₂. Hydrogenation of 4-octyne was chosen as a model catalytic reaction for hydrogenation (see Scheme 1 below).

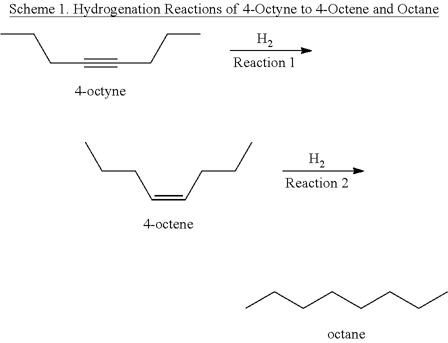

The desired product in our study is 4-octene and selectivity is defined as the ratio [4-octene]/([4-octene]+[octane]).

Thoroughly washed Pt and CoPt₃ NPs showed high activity for hydrogenation reactions leading to the complete conversion of 4-octyne into octane (see FIG. 1c—light gray) which means virtually 0% selectivity for 4-octene (see FIG. 1c). However, the addition of primary alkylamine to the reaction mixture had a major effect on the reaction products. Introduction of octylamine switched the selectivity for the desired 4-octene (see FIG. 1c—dark gray) to a value greater than 90%; and the apparent conversion was at the same level as for purified NPs (about 99.9% conversion, as shown in FIG. 1c). Among the isomers of 4-octene, the cis form was dominant in the product. Controlled experiments using 6 nm Co NPs and 1-octylamine showed zero activity for the alkyne hydrogenation reaction. Thus, the observed substantial enhancement in the selectivity of Pt and CoPt nanocatalysts in the presence of octylamine was a consequence of surface modification of these NPs with octylamine.

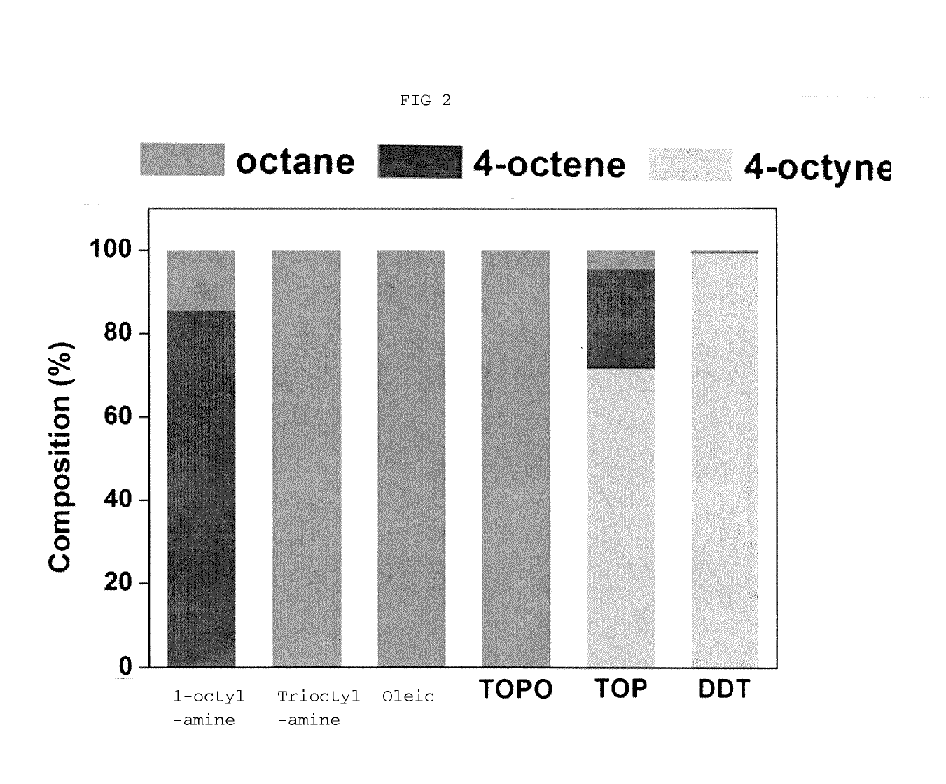

In another evaluation, different capping ligands rather than primary amines were added that are used in the synthesis of NPs to 4-octyne solutions containing purified 3.5 nm Pt NPs; and their effect was determined on the catalytic performance. Addition of trioctylamine, oleic acid, and trioctylphosphine oxide (TOPO) affected neither the selectivity nor the activity as compared with the “clean” Pt NPs. Addition of trioctylphosphine (TOP) significantly lowered the activity, while 1-dodecanethiol (DDT) almost completely deactivated Pt NPs (see FIG. 2). The hydrogenation of alkynes proceeds through sequential addition of H₂, to π-bonding orbitals of unsaturated hydrocarbons (see the Scheme 1 table). These reactions take place on the surfaces of the NP catalysts. The effect of octylamine shown in FIGS. 1a-1c and 2 suggests that among various capping ligands tested, primary alkylamines allow for Reaction 1 but block Reaction 2 (again see Scheme 1).

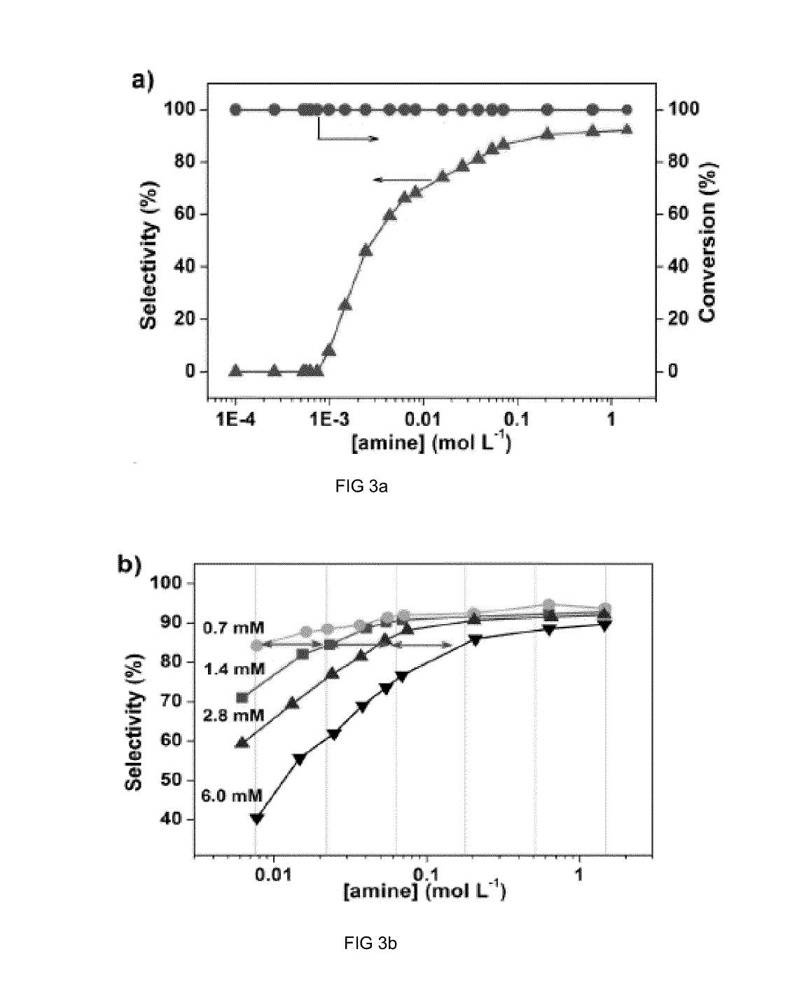

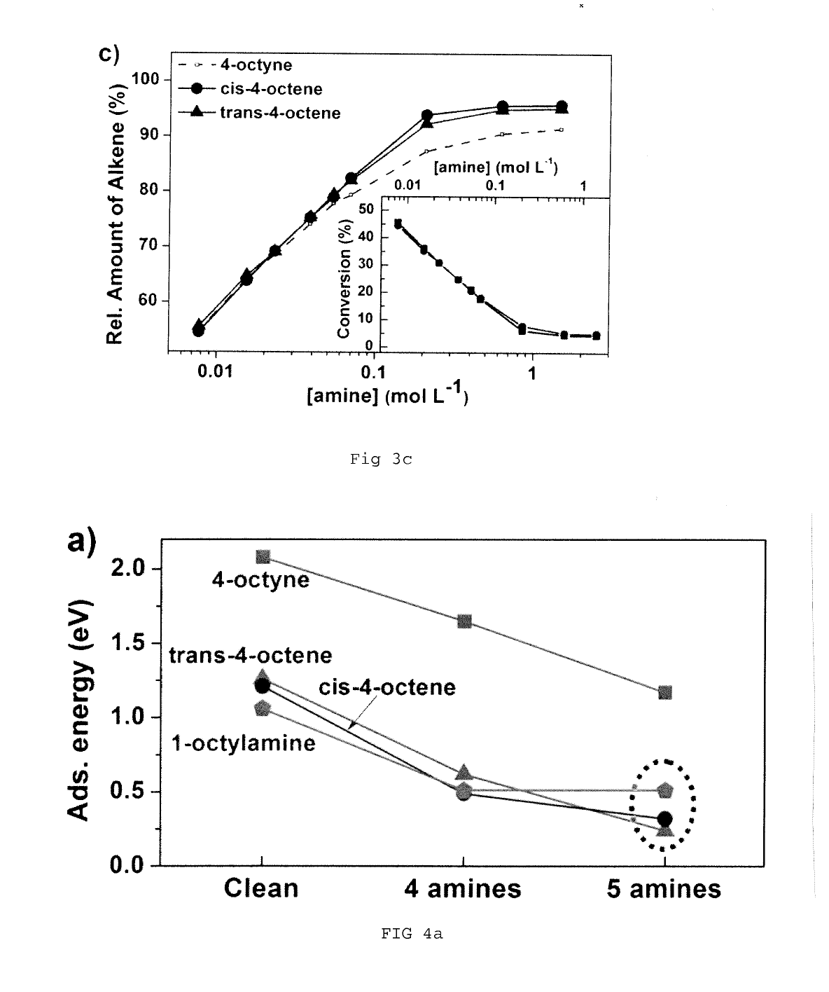

To obtain a better understanding of the role of primary amines in alkyne hydrogenation catalyzed by Pt NPs, the effect of surface coverage of catalyst by the primary amines was analyzed and selectivity determined. The degree of amine surface coverage by the concentration of the amines in the reaction mixture was carefully measured since there is a straightforward correlation between the concentrations of added amines and adsorbed at the surface of NPs. The octylamine concentration dependence of selectivity (see FIG. 3a) is thereby characterized. The onset of selectivity starts at the octylamine concentration of about 1-2 mM, corresponding to a monolayer or slightly thicker coverage of the NP surface with amines if all the added octylamine molecules would adsorb onto the NP surface. That is not necessarily the case since there is a dynamic equilibrium between octylamine molecules on the NP surface and in the solution. Further increase in the octylamine concentration resulted in a rapid increase of selectivity that saturated at the octylamine concentration of about 100 mM. The total level of conversion of 4-octyne was more than 99.9% at any concentration of octylamine. These data indicated that in a preferred embodiment the selectivity was defined by the degree of surface coverage with the amines. Data supporting the central role of amine coverage are also presented in FIG. 3b, which shows dependence of selectivity as a function of the amine concentration for four different concentrations of Pt NPs. In this experiment, the total (combined) surface area of Pt NPs changes from case to case by a factor of about two. Inspection of the graphs shows that four cases exhibit the same selectivity at amine concentrations but also differ from one case to another by a factor of about two. In other words, the amine concentrations that furnish a given level of selectivity are generally proportional to the total surface area of the NPs.

Without limiting the scope of the invention, the data in FIGS. 3a and 3b, indicate that the high coverage of amine at the surface of NPs results in high selectivity for 4-octene by blocking the hydrogenation of 4-octene to octane (see Reaction 2 in Scheme 1). In order to test this idea, hydrogenation reactions were performed of cis- and trans-forms of 4-octene as starting reagents. The increase in amine concentration results in the blocking of 4-octene hydrogenation into octane (see FIG. 3c) leaving nearly the same amount of 4-octene as in the case of 4-octyne hydrogenation at the same concentrations of octylamine (see FIG. 3a). The relative amount of alkene after cis- or trans-4-octene hydrogenation was slightly (about 5%) higher than that from 4-octyne hydrogenation. This difference can be attributed to a small fraction of 4-octyne which underwent a direct complete hydrogenation into 4-octane. Again without limiting the scope of the invention, the experimental data presented in FIGS. 3a-3c provide evidence for the role of primary alkylamines as selectivity switchers/tuners. This role is believed to attenuate or even block almost completely the hydrogenation of 4-octene.

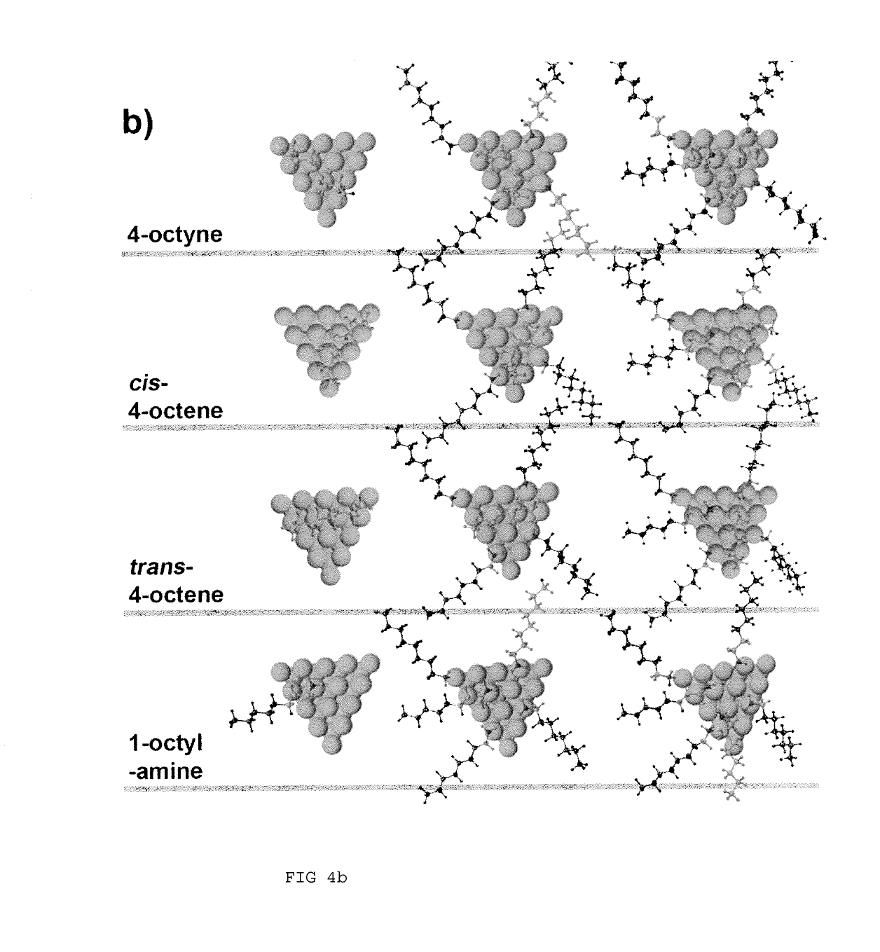

In order to uncover the mechanism by which the amine ligands affect the selectivity in catalytic hydrogenation of alkynes, DFT studies were performed of the energetics relevant to the processes at hand. In particular, the adsorption energies were evaluated for 4-octyne, cis-/trans-4-octene, and octylamine on the same corresponding sites of a clean and octylamine-covered (111) surface of Pt as represented by a 35-atom Pt cluster. The DFT computations showed that 4-octyne has the highest affinity to the clean surface of Pt while alkylamine has the lowest (see FIGS. 4a and 4b, and Example VII). FIGS. 4a and 4b also clearly show that as the number of the coadsorbed octylamines increases, the adsorption energies of all four species decrease. Increasing the octylamine coverage on Pt surface reduces the adsorption energy of amines, alkenes, and alkynes due to the steric interactions between hydrocarbon chains. At all coverages, the adsorption energy of 4-octyne is higher than those of either form of 4-octene or octylamine. Notably, higher coverage of amine on the Pt surface affects the adsorption energy of species with C═C double bonds the most (see FIG. 4a). At sufficiently high coverage of Pt surface with amines, the change in the order of adsorption energies weighs the competition between 4-alkenes and octylamines for binding sites on the catalyst surface in favor of amines (see FIG. 4a). At high concentrations of amine in the solution, the coverage of amine on the surface of the Pt NPs increases so that the adsorption energy of amines becomes higher than those of alkenes and, as a result, the adsorption of alkene molecules is impeded, which prevents their further conversion into octanes (see Scheme 1). On the other hand, the adsorption energy of 4-octyne is still substantially higher than that of primary amine even at the high coverage of amine and, hence, the hydrogenation of 4-octyne to 4-octene is not impeded at any amine concentration. Again without limiting the scope of the invention, the coverage-dependent trends and changes in the adsorption energies of substrates and capping ligands provide the rationale for understanding the role of primary amines as selectivity switches in catalytic hydrogenation of alkynes on Pt nanoparticles.

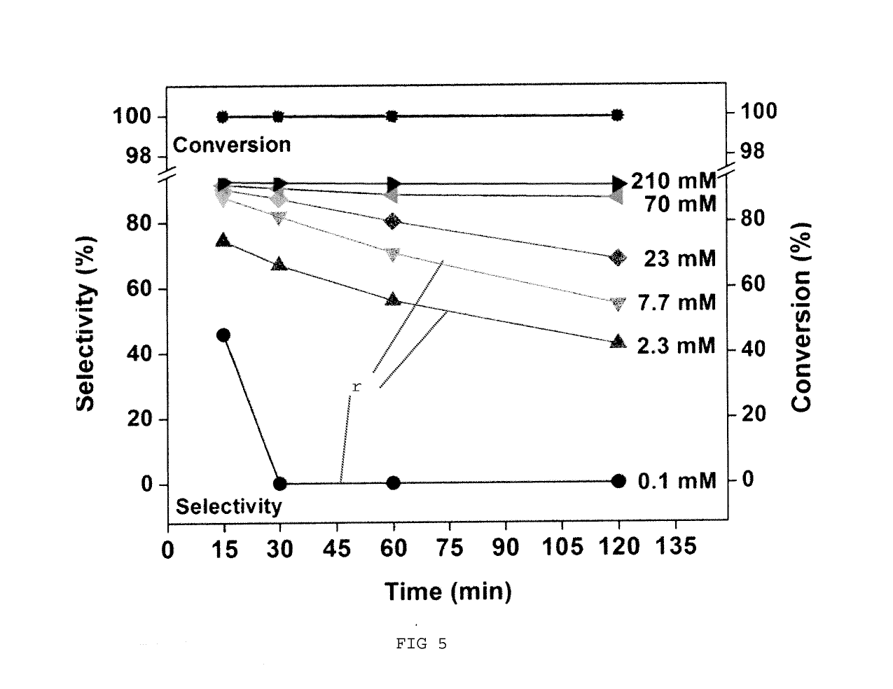

The above recited observation is also consistent with the results of kinetic measurements shown in FIG. 5. The activity (conversion of 4-octyne) is about 99.9% over all time scales considered. The selectivity, however, exhibits different patterns of time-dependence at different amine concentrations. At the lowest concentration ofoctylamine (0.1 mM), the selectivity of about 45% at t=15 min drops to 0% at t=30 min meaning that all 4-octyne is converted into octane. At the highest amine concentration (210 mM), the selectivity is above 90% and negligibly changes with time. This is consistent with the displacement of all 4-octenes from the catalyst surface by octylamines as soon as 4-octenes are generated from 4-octynes. At intermediate octylamine concentrations, the initial high selectivities show a gradual decline with time. The rate of this decline decreases as the octylamine concentration increases (see FIG. 5); and this correlates with the decrease in the 4-octene adsorption energy as the octylamine coverage of the Pt nanocatalyst increases (see FIGS. 4a and 4b). Thus the theoretical calculations are in very good agreement with the experimental results shown in FIGS. 3-5 and explain high selectivity and full conversion of alkynes at the surface of nanocatalysts densely covered with molecules of primary alkylamines.

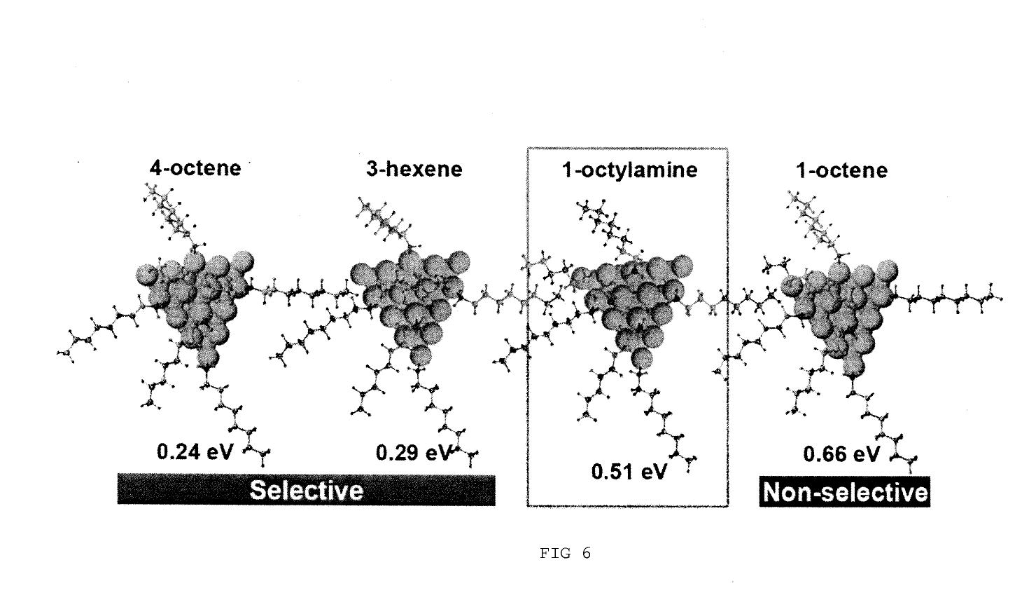

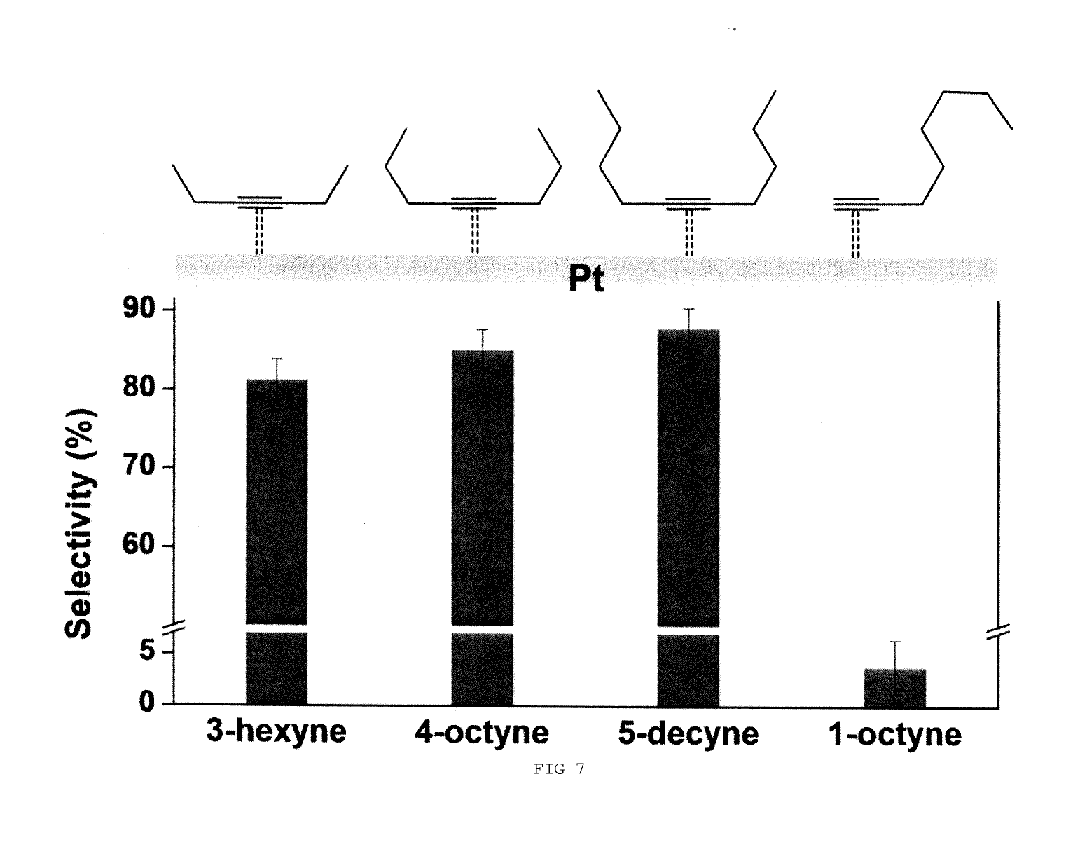

As mentioned above, DFT calculations indicate that the adsorption energetics of alkenes (CₙH₂ₙ, n=3, 4) compared to that of amines defines hydrogenation selectivity. Our computational results show that at high coverage of Pt surface with octylamine the adsorption energies of 4-octene and 3-hexene are lower than that of octylamine while 1-octene has a higher adsorption energy (see FIG. 6). As a consequence we can expect high yield of alkene in hydrogenation reactions of 4-octyne and 3-hexyne and a high degree of conversion of 1-octyne into octane. Indeed, experimentally, alkene selectivities greater than 80% were observed in hydrogenation reactions of 3-hexyne, 4-octyne, and 5-decyne; however, selectivity of only about 4% was observed in the case of 1-octyne hydrogenation (see FIG. 7).

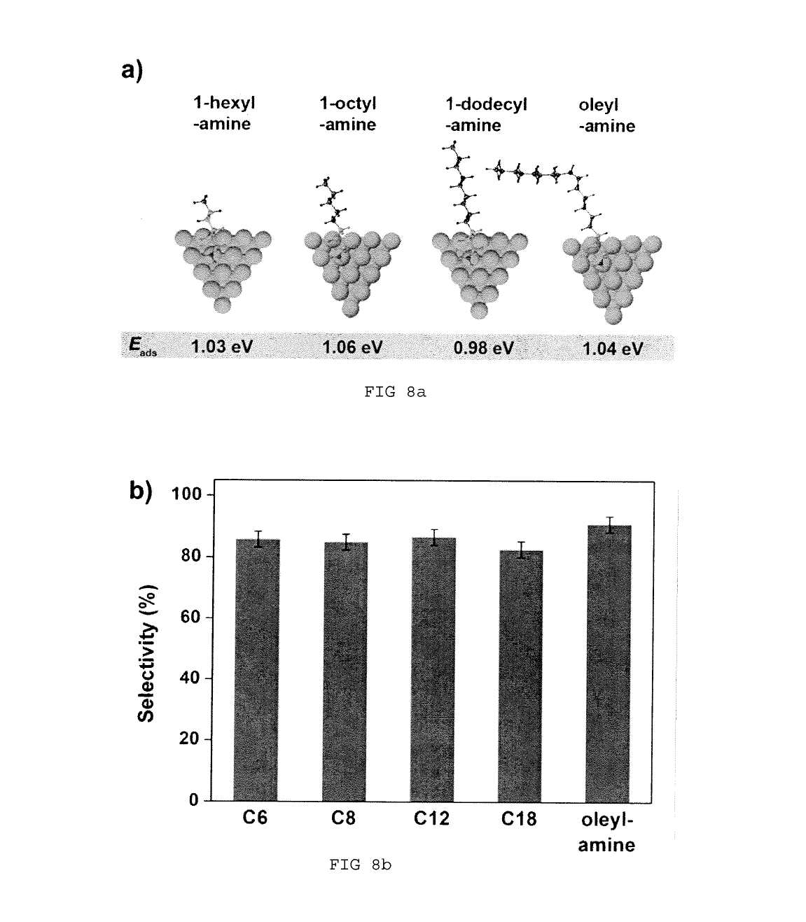

In order to provide further insight into selective exclusion of alkene from the surface of Pt NPs by the primary amines, the eftfict was considered of the hydrocarbon chain length of amine on the selectivity in 4-octyne hydrogenation reaction. Our DFT calculations revealed that primary alkylamines with different chain lengths have approximately the same adsorption energy on Pt (see FIG. 8a) and, therefore, they can be expected to have a similar effect on selectivity. The experiments corroborate this prediction. Experimentally, as in the case of octylamine, the same high selectivity (in the range between about 83 and 91%) was observed for all studied primary alkylamines (see FIG. 8b).

Although not limiting the scope of the invention, the DFT calculations also provide a rational explanation for the observed low performance of trioctylamine as a selectivity promoter and for TOP being an activity attenuator (see FIG. 2). The calculated adsorption energy of trioctylamine on Pt is 0.84 eV, which is lower than those of the primary amines, while the adsorption energy of TOP is 2.55 eV, which is higher than that of even 4-octyne. The lower adsorption energy of trioctylamine makes it a less favorable candidate for substitution of 4-octene. The high adsorption energy of TOP makes it a “poison” for the catalyst. These findings and considerations point to the importance of adsorption energy as a guiding parameter for a judicious selection of capping ligands with desired activity and selectivity effects.

Our combined experimental/computational evaluations on capping ligands as modifiers of catalytic functionality of nanoparticles provided advantageous results; and a methodology is also provided for understanding the role of surface modification in defining/affecting the catalysts' activity and/or selectivity. In particular, the role of particular capping ligands is significant as a means for substantial enhancement of selectivity. Using Pt and CoPt₃, NPs as catalysts, hydrocarbons and hydrogen as reactants, and alkylamines, oleic acid, trioctylphos trioctylphosphine, and trioctylphosphine oxide as capping ligands, the balance between the adsorption energetics of substrates and capping ligands can determine the selectivity and activity of catalyst. A judicious selection of capping ligands with appropriate adsorption energies on catalyst can lead to a substantial and advantageous enhancement in its selectivity. Sufficient concentrations of primary alkylamine ligands can result in higher than 90% selectivity toward selective hydrogenation at an overall activity of 99.9%. Capping ligands with too low adsorption energies, as compared to those of the substrates, have little or no effect on selectivity. Capping ligands with too high adsorption energies significantly reduce activity.

The following non-limiting examples provide various exemplary information on preparation and analysis of selected catalysts.

### Example I

### Methodological Details

### Synthesis of NPs

The synthesis of Pt NPs was carried out by a method described in conventional prior art with minor modification. A reaction mixture was prepared by adding 0.2 g of Pt(acac)₂, 0.89 g of oleic acid, and 0.81 g of oleylamine into 10 mL of 1-octadecene. It was degassed at 100° C. for 20 min and heated at 120° C. for 30 min under nitrogen atmosphere to form a clear yellow solution. It was further heated to 200° C. at the rate of 4° C. min and then kept at that temperature for 30 min. After the reaction was stopped, Pt NPs were separated and washed with excess acetone two times. CoPt₃, NPs were synthesized using conventional well-known methodologies.

### Example II

### Catalytic Studies

The hydrogenation reaction was carried out in a stainless steel reactor at room temperature for 3 h under H₂ atmosphere (200 psig). In a standard condition, the reaction solution was prepared by dispersing Pt nanoparticles in 1.0 mL dodecane containing 3.75 wt % (255 mM) of 4-octyne. The amount of Pt in the solution was controlled in the range of 0.4-0.6 mg, which was confirmed by inductively coupled plasma (ICP) analysis. [Pt]_{surf} is defined as the concentration of Pt atoms at the surface of Pt NPs in the solution. The value of [Pt]_{surf} is calculated based on the net amount of Pt atoms in the solution and the size of Pt NPs from TEM. During the reaction, the solution was stirred at about 7000 rpm. After the reaction, the solutions were purged with nitrogen to remove any residual hydrogen. Otherwise stated, the concentration of amine in the reaction solution was controlled by adding 1-octylamine. On the basis of the composition data from the reaction product, conversion and selectivity of the catalytic reaction of 4-octyne are defined as follow: (Conversion)=1−[4-octyne]/[4-octyne]ᵢₙᵢₜᵢₐₗ; (Selectivity)=[4-octene]/([4-octene]+[octane]).

### Example III

### Characterization

Samples for transmission electron microscopy (TEM) were prepared by dropping and drying of 1-2 pL of toluene solution of NPs on a carbon-coated copper grid (Ted Pella). TEM measurements were performed using a JEOL 2100F microscope operated at 200 kV. Thermogravimetric analysis was carried out using a Mettler Toledo TGA/SDTA851e instrument. The sample was heated from 25 to 600° C. at the heating rate of 3° C./min. The composition of the solution after hydrogenation reaction was analyzed by gas chromatography mass spectrometry (GC-MS) instrument composed of an Agilent 6890 GC system and a 5973 Network Selective Detector.

### Example IV

### Computational Framework

The computations were performed within density functional theory with the PBE exchange-correlation functional, double-ζ basis sets, and Goedecker—Teter—Hutter type pseudo potentials as implemented in the CP2K package. The size of the computation cell was 30×30×30 Å. (See also Example VII).

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

### Example V

In our competitive binding model reaction, selectivity for 4-octene is determined by the hydrogenation reaction rate of 4-octene into octane (FIG. 5). The rate of the catalytic hydrogenation reaction is proportional to the number of substrate molecules adsorbed onto the active site of the catalyst surface. When alkylamine and octene molecules coexist on the surface, the adsorption equilibrium between them can be described by the Langmuir isotherm equation as follow:

$${θ}_{octene} = \frac{[ octene ]}{1 + {K}_{amine} [ amine ] + {K}_{octene} [ octene ]} \qquad ( 1 )$$

In the above equations, θ_{octene} is the surface coverage of the adsorbed 4-octene and Kₐₘᵢₙₑ and K_{octene} are the adsorption coefficients. [amine] and [octane] are the concentration of free amine and 4-octene molecules in the solution. Because the faster hydrogenation of 4-octene leads to the lower selectivity, selectivity should be proportional to (1−θ_{octene}) approximately. By rearranging Eq. (1), we get an expression for (1−θ_{octene}) as follow.

$$( 1 - {θ}_{octene} ) = \frac{[ amine ]}{{K}_{octene} [ octene ] / {K}_{amine} + [ amine ]} \qquad ( 2 )$$

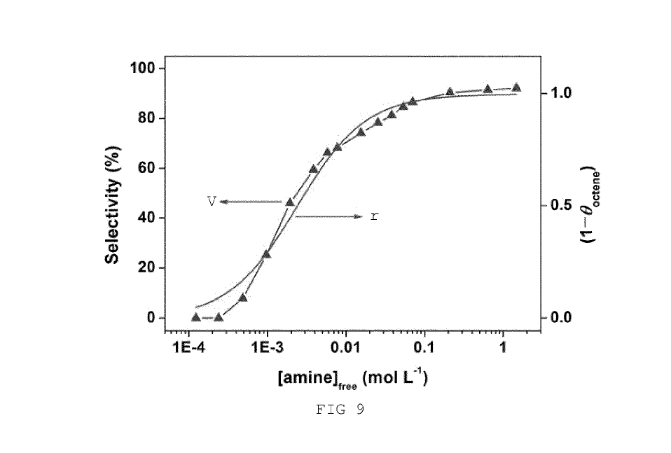

In the model reactions, the concentration of surface binding amine is in the range of 0.5-0.7 mM. In FIG. 9, the selectivity curve in FIG. 3a is re-plotted according to the concentration of free amine in the solution. Using eq S2, we fitted the selectivity curve and the best fit result was obtained when K_{octene}/Kₐₘᵢₙₑ˜0.01. As shown in the figure, the fit curve for (1−θ_{octene}) shows good resemblance to the selectivity curve. This data strongly supports that the dependence of selectivity on amine concentration is determined by the competitive adsorption between 4-octene and amine.

### Example VI

To study the chemical stability of the NP catalysts, we carried out extended X-ray absorption fine structure (EXAFS) measurements on CoPt. NPs before and after the hydrogenation reaction (Tables 1 and 2 below). The sample description is as follow.
Before & After: CoPt₁ NPs before and after 4-octyne hydrogenation reaction with the presence of 1-octylamine.
No substrate: CoPt₃ NPs treated in the same condition as for hydrogenation reaction but no substrate molecules.
   The data from the samples is very similar to that of the foil which suggests that Pt atoms in the samples have local structure very similar to that of metallic platinum. Unlike Pt, the Co edge spectra from the samples have very less similarity with Co atoms in Co metal or Co oxide which is possibly due to the presence of large number of Pt atoms in the neighborhood of Co atoms. The samples from before and after the reaction show little structural changes except for the absence of Co—O bonding for after-reaction sample.

TABLE 1
List of fit parameters obtained from modeling Co edge data of CoPt₃ samples. Sₒ² has
been calculated from Co foil data and has been set to 0.8 for Co edge data analysis.

| | | | | | Debye | Energy |
|---|---|---|---|---|---|---|
| | | | Bond length | Coordination | Waller | Shift |
| Edge | Sample Name | Paths | R (ang) | Number (n) | Factor | ΔE (eV) |
| Co | Before | Co—O | 2.02 ± 0.05 | 1.4 ± 0.6 | 0.009 [symbol] | −4.3 ± 2.4 |
| | k = 2.5-8 Å⁻¹ | Co—Co | 2.57 ± 0.01 | 1.0 ± 0.9 | 0.01 ± 0.003 | |
| | R = 1.2-3.6 Å | (metal) | | | | |
| | | Co—Pt | 2.70 ± 0.01 | 6.9 ± 1.7 | 0.01 ± 0.003 | |
| | After | Co—Co | 2.57 ± 0.01 | 0.9 ± 0.6 | 0.01 ± 0.002 | −5.1 ± 0.1 |
| | k = 2.5-9.0 Å⁻¹ | (metal) | | | | |
| | R = 1.2-3.6 Å | Co—Pt | 2.70 ± 0.01 | 7.3 ± 1.2 | 0.01 ± 0.002 | |
| | No substrate | Co—Co | 2.56 ± 0.01 | 0.5 ± 0.4 | 0.01 ± 0.001 | −5.5 ± 0.8 |
| | k = 2.5-10 Å⁻¹ | (metal) | | | | |
| | R = 1.2-3.6 Å | Co—Pt | 2.69 ± 0.01 | 7.1 ± 0.9 | 0.01 ± 0.001 | |

This SS value has been taken from Co—O bond by fitting Co edge from CoO compound. [symbol]
indicates data missing or illegible when filed

TABLE 2
List of fit parameters obtained from modeling Pt edge data of CoPt3 samples. Sₒ² has
been calculated from Pt foil data and has been set to 0.84 for Pt edge data analysis.

| | | | | | Debye | Energy |
|---|---|---|---|---|---|---|
| | | | Bond length | Coordination | Waller | Shift |
| Edge | Sample Name | Paths | R (ang) | Number (n) | Factor | ΔE (eV) |
| Pt | Before | Pt—Pt1 | 2.77 ± 0.01 | 8.4 ± 1.2 | 0.003 ± 0.002 | −4.3 ± 2.4 |
| | k = 3.0-8.6 Å⁻¹ | Pt—Pt2 | 3.88 ± 0.02 | 4.8 ± 1.5 | 0.003 ± 0.002 | |
| | R = 1.2-4 Å | Pt—O | 2.01 ± 0.02 | 0.9 ± 0.2 | 0.004 [symbol] | |
| | After | Pt—Pt1 | 2.76 ± 0.01 | 10.5 ± 2.5 | 0.006 ± 0.003 | −5.1 ± 0.1 |
| | k = 3-8.6 Å⁻¹ | Pt—Pt2 | 3.88 ± 0.04 | 5.7 ± 2.9 | 0.006 ± 0.003 | |
| | R = 1.2-4 Å | Pt—O | 2.1 ± 0.08 | 0.4 ± 0.3 | 0.004 | |
| | No substrate | Pt—Pt1 | 2.77 ± 0.02 | 9.8 ± 2.9 | 0.006 ± 0.003 | −5.5 ± 0.8 |
| | | Pt—Pt2 | 3.90 ± 0.04 | 7.0 ± 4.0 | 0.006 ± 0.003 | |
| | | Pt—O | 2.06 ± 0.04 | 1.1 ± 0.4 | 0.004 | |

This SS value taken from Pt—O bond by fitting Pt data from PtO₂. [symbol]
indicates data missing or illegible when filed

### Example VII

The adsorption energy of the ligands (alkynes, alkenes or amines) on the bare cluster is computed as

E_{Ads}=E_{cluster}+E_{X}−E_{Total} (3)

where E_{cluster}, E_{X} and F_{Total} are the equilibrium energies of the bare cluster, the ligand molecule in the gas phase, and the cluster-ligand complex, respectively.

The adsorption energy of the ligands (alkynes, alkenes or amines) on the cluster functionalized by “surrounding” amines is computed as

E_{Ads}=E_{cluster+amines}+E_{X}−E_{Total} (4)

where E_{cluster+amines}, E_{X} and E_{Total} are the equilibrium energies of the amine-functionalized cluster, the ligand molecule in the gas phase, and the functionalized cluster-ligand complex, respectively.

The foregoing description of embodiments of the present invention have been presented for purposes of illustration and description. It is not intended to be exhaustive or to limit the present invention to the precise form disclosed, and modifications and variations are possible in light of the above teachings or may be acquired from practice of the present invention. The embodiments were chosen and described in order to explain the principles of the present invention and its practical application to enable one skilled in the art to utilize the present invention in various embodiments, and with various modifications, as are suited to the particular use contemplated.

What is claimed is:

1. A method of manufacturing a catalyst, comprising the steps of,
providing noble metal based particles; and
in a reaction chamber exposing the noble metal based catalyst to a reaction mixture including an amine, thereby forming the catalyst.

2. The method as defined in claim 1 wherein the amine comprises a primary alkylamine which thereby selectively prevents further hydrogenation of alkenes in a hydrogenation reaction.

3. The method as defined in claim 2 wherein the primary amine is selected from the group consisting of 1-hexylamine, 1-octylamine, 1-dodecylamine, 1-octadecylamine, oleyl-amine and trioctyl-amine.

4. The method as defined in claim 2 further including the step of establishing a minimum effective concentration of the primary alkyl amine in the reaction mixture, thereby establishing onset of selectivity for hydrogenation of a feedstock input to the catalyst.

5. The method as defined in claim 4 wherein the minimum effective concentration is about 1-2 mM octylamine concentration in the reaction mixture.

6. The method as defined in claim 4 wherein the minimum effective concentration corresponds to forming at least a surface monolayer of the primary alkylamine on the catalyst.

7. The method as defined in claim 4 further including processing the feedstock wherein the feedstock comprises 4-octyne.

8. The method as defined in claim 1 wherein the noble metal based catalyst is selected from the group of Pt, CoPt₃ and Pd.

9. The method as defined in claim 1 wherein the noble metal based particles comprises nanoparticles.

10. An article of manufacture of a catalyst, comprising:
a noble metal based catalyst material having a primary alkylamine coating thereon.

11. The article of manufacture as defined in claim 10 wherein the noble metal catalyst comprises at least one of a Pt containing material and a Pd containing material.

12. The article of manufacture as defined in claim 11 wherein the Pt containing material is selected from the group of Pt and alloys of Pt.

13. The article of manufacture as defined in claim 10 wherein the noble metal based catalyst material comprises a nanoparticle material.

14. The article of manufacture as defined in claim 10 wherein the primary alkylamine is selected from the group of 1-hexylamine, 1-octylamine, 1-dodecyclamine, 1-octadecylamine, oleyl-amine and trioctyl-amine.

15. The article of manufacture as defined in claim 10 wherein the primary alkylamine coating comprises at least one monolayer on a surface of the noble metal based catalyst.

16. A method of performing a hydrogenation process of a hydrocarbon feedstock, comprising the steps of,
obtaining a noble metal based catalyst by (1) providing noble metal containing particles and (2) in a reaction chamber exposing the noble metal containing particles to a primary alkylamine to obtain a product catalyst; and
flowing a feedstock over the product catalyst to hydrogenate the hydrocarbon feedstock to form a selectively hydrogenated product.

17. The method as defined in claim 16 wherein the noble metal based catalyst comprises a nanoparticle material.

18. The method as defined in claim 17 wherein the primary alkylamine forms at least a monolayer on a surface of the nanoparticle material.

19. The method as defined in claim 16 wherein the primary alkylamine is selected from the group of 1-hexylamine, 1-octylamine, 1-dodecylamine, 1-octadecylamine, oleyl-amine and trioctyl-amine.

20. The method as defined in claim 16 wherein the noble metal based catalyst includes at least one of Pt and Pd.
